(19) United States
(12) Patent Application Publication
(10) Pub. No.: US 2020/0035361 A1
(43) Pub. Date: Jan. 30, 2020

(54) METHOD AND ELECTRONIC DEVICE FOR ARTIFICIAL INTELLIGENCE (AI)-BASED ASSISTIVE HEALTH SENSING IN INTERNET OF THINGS NETWORK

(71) Applicant: Samsung Electronics Co., Ltd, Suwon-si (KR)

(72) Inventors: Ilavarasu Jayabalan ELLAN, Bangalore (IN); Ariyalur Chandrasekaran GANESH, Bangalore (IN); Rames PALANISAMY, Bangalore (IN)

(21) Appl. No.: 16/523,835

(22) Filed: Jul. 26, 2019

(30) Foreign Application Priority Data
Jul. 26, 2018 (IN) 201841028156
Jul. 12, 2019 (IN) 2018 41028156

(51) Int. Cl. G16H 50/20; G16H 40/20

(52) U.S. Cl. CPC G16H 50/20; H04L 67/12; G16H 40/20

(57) ABSTRACT

Embodiments herein disclose a method for AI-based assistive health sensing in an IoT network comprising a plurality of electronic devices connected with each other. The method includes obtaining, by a first electronic device from the plurality of electronic devices, at least one input indicating a current health condition of a user. Further, the method includes determining, by the first electronic device, at least one vital parameters of the user to be measured based on the current health conditions of the user using at least one AI model. Further, the method includes identifying, by the first electronic device, at least one second electronic device from the plurality of electronic devices to measure the at least one vital parameter for the user using the at least one AI model. Further, the method includes automatically initiating, by the first electronic device, a conversation with the user.

### CROSS-REFERENCE TO RELATED APPLICATIONS

This application is based on and claims priority under Indian Patent Application Number 201841028156 filed on Jul. 26, 2018 and Indian Patent Application Number 201841028156 filed on Jul. 12, 2019, the disclosures of which are herein incorporated by reference in their entirety.

### BACKGROUND

### 1. Field

Currently, a user needs to call to schedule an appointment for a medical consultation or a health checkup. Further, the user needs to visit a hospital at the appointment time to meet a care taker (e.g., physician, doctor, or the like).

### 2. Description of Related Art

In case if the user selects an online medical consultation then, it is hard for the user to explain a health history and sharing the different Electronic Health Records (EHRs) during the online medical consultation with the care taker for a specific condition (e.g., chronic disease, or the like). It may be possible that hidden insights might be missed during the online medical consultation with the care taker.

Thus, it is desired to address the above mentioned disadvantages or other shortcomings or at least provide a useful alternative.

### SUMMARY

The present disclosure relates to a healthcare management system, and more specifically related to a method and electronic device for artificial intelligence (AI)-based assistive health sensing in an internet of things (IoT) network based on a vital parameter.

Accordingly, the embodiments herein disclose a method for AI-based assistive health sensing in an IoT network including a plurality of electronic device connected with each other. The method includes obtaining, by a first electronic device from the plurality of electronic devices, at least one input indicating a current health condition of a user. Further, the method includes determining, by the first electronic device, at least one vital parameters of the user to be measured based on the current health conditions of the user using at least one AI model. Further, the method includes identifying, by the first electronic device, at least one second electronic device from the plurality of electronic devices to measure the at least one vital parameter for the user using the at least one AI model. Further, the method includes automatically initiating, by the first electronic device, a conversation with the user. The conversation includes an operating guidance to measure the at least one vital parameter of the user using the at least one second electronic device.

In an embodiment, identifying, by the first electronic device, the at least one second electronic device from the plurality of electronic devices to measure the at least one vital parameter for the user using the at least one AI model includes determining, by the first electronic device, a capability of each of the electronic devices connected to the first electronic device, and identifying, by the first electronic device, the at least one second electronic device to measure the at least one vital parameter for the user based on the capability of each of the electronic devices.

In an embodiment, further, the method includes obtaining, by the first electronic device, the at least one measured vital parameter using the at least one second electronic device for the user. Further, the method includes analyzing, by the first electronic device, the measured vital parameters. Further, the method includes recommending, by the first electronic device, a caretaker related to the current health condition of the user for appointment based on the analysis.

In an embodiment, further, the method includes receiving, by the first electronic device, a health care instruction from the caretaker based on the at least one vital parameter. Further, the method includes monitoring, by the first electronic device, the health care instruction based on the at least one vital parameter.

Accordingly, the embodiments herein disclose a method for AI-based assistive health sensing in an IoT network including a plurality of electronic device connected with each other. The method includes obtaining, by a first electronic device from the plurality of electronic devices, at least one input indicating a current health condition of a user. Further, the method includes automatically booking, by the first electronic device, an appointment with a caretaker related to the current health condition of the user. Further, the method includes automatically initiating, by the first electronic device, a conversation with the user to measure the at least one vital parameter at a predetermined time prior to the appointment with the caretaker.

In an embodiment, further, the method includes determining, by the first electronic device, a capability of each of the electronic devices connected to the first electronic device. Further, the method includes identifying, by the first electronic device, the at least one second electronic device to measure the at least one vital parameter for the user based on the capability of each of the electronic devices using at least one AI model. Further, the method includes initiating, by the first electronic device, the conversation comprising an operating guidance to measure the at least one vital parameter of the user using the at least one second electronic device.

In an embodiment, further, the method includes obtaining, by the first electronic device, the at least one measured vital parameter using the at least one second electronic device for the user. Further, the method includes sharing, by the first electronic device, the at least one measured vital parameter with the caretaker prior to the appointment.

In an embodiment, automatically booking, by the first electronic device, the appointment with the caretaker related to the current health condition of the user includes recommending by the first electronic device, the caretaker related to the current health condition of the user, receiving, by the first electronic device, a confirmation from the user for the appointment with the caretaker, and booking, by the first electronic device, the appointment with the caretaker.

Accordingly, the embodiments herein disclose an electronic device for AI-based assistive health sensing in an IoT network comprising a plurality of electronic device connected with each other. The electronic device includes a processor coupled with a memory. The processor is configured to obtain at least one input indicating a current health condition of a user. Further, the processor is configured to determine at least one vital parameters of the user to be measured based on the current health conditions of the user using at least one AI model. Further, the processor is configured to identify at least one another electronic device from the plurality of electronic devices to measure the at least one vital parameter for the user using the at least one AI model. Further, the processor is configured to automatically initiate a conversation with the user. The conversation includes an operating guidance to measure the at least one vital parameter of the user using the at least one another electronic device.

Accordingly, the embodiments herein disclose an electronic device for AI-based assistive health sensing in an IoT network comprising a plurality of electronic device connected with each other. The electronic device includes a processor coupled with a memory. The processor is configured to obtain at least one input indicating a current health condition of a user. Further, the processor is configured to automatically book an appointment with a caretaker related to the current health condition of the user. Further, the processor is configured to automatically initiate a conversation with the user to measure the at least one vital parameter at a predetermined time prior to the appointment with the caretaker.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1A illustrates overview of an IoT network comprising a plurality of electronic device for AI-based assistive health sensing, according to the embodiments as disclosed herein;

FIG. 1B illustrates another overview of the IoT network comprising the plurality of electronic device and a server for the AI-based assistive health sensing, according to the embodiments as disclosed herein;

FIG. 2A illustrates various hardware blocks of an electronic device, according to the embodiments as disclosed herein;

FIG. 2B illustrates various hardware blocks of the processor included in the electronic device, according to the embodiments as disclosed herein;

FIG. 3A illustrates various hardware blocks of a server, according to the embodiments as disclosed herein;

FIG. 3B illustrates various hardware blocks of a processor included in the server, according to the embodiments as disclosed herein;

FIG. 4 illustrates a flow chart for automatically initiating a conversation, with a user, comprising an operating guidance to measure the at least one vital parameter of the user, according to the embodiments as disclosed herein;

FIG. 5 illustrates a flow chart for automatically initiating a conversation with the user to measure at least one vital parameter at a predetermined time prior to an appointment with a caretaker, according to the embodiments as disclosed herein;

FIG. 6 illustrates are example sequence diagram including various operations for providing a care plan management based on the AI-based assistive health sensing, according to the embodiments as disclosed herein; and

FIG. 7 illustrates an example in which the electronic device provides health care decision, according to the embodiments as disclosed herein.

### DETAILED DESCRIPTION

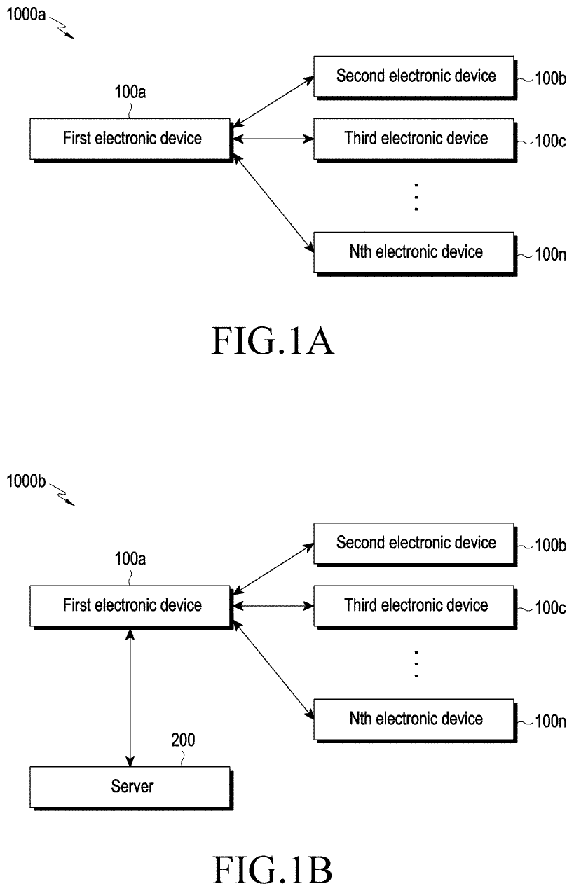

FIGS. 1A through 7, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

The principal object of the embodiments herein is to provide a method and system for AI-based assistive health sensing in an IoT network.

Another object of the embodiments herein is to obtain, by a first electronic device from a plurality of electronic devices, at least one input indicating a current health condition of a user.

Another object of the embodiments herein is to determine, by the first electronic device, at least one vital parameters of the user to be measured based on the current health conditions of the user using at least one AI model.

Another object of the embodiments herein is to identify, by the first electronic device, at least one second electronic device from the plurality of electronic devices to measure the at least one vital parameter for the user using the at least one AI model.

Another object of the embodiments herein is to automatically initiate, by the first electronic device, a conversation with the user, where the conversation includes an operating guidance to measure the at least one vital parameter of the user using the at least one second electronic device.

Another object of the embodiments herein is to recommend, by the first electronic device, a caretaker related to the current health condition of the user for appointment.

Another object of the embodiments herein is to receive, by the first electronic device, a health care instruction from the caretaker based on the at least one vital parameter.

Another object of the embodiments herein is to automatically book, by the first electronic device, an appointment with the caretaker related to the current health condition of the user.

Another object of the embodiments herein is to automatically initiate, by the first electronic device, the conversation with the user to measure the at least one vital parameter at a predetermined time prior to the appointment with the caretaker.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks which carry out a described function or functions. These blocks, which may be referred to herein as managers, units, modules, hardware components or the like, are physically implemented by analog and/or digital circuits such as logic gates, integrated circuits, microprocessors, microcontrollers, memory circuits, passive electronic components, active electronic components, optical components, hardwired circuits and the like, and may optionally be driven by firmware and software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the disclosure. Likewise, the blocks of the embodiments may be physically combined into more complex blocks without departing from the scope of the disclosure.

The accompanying drawings are used to help easily understand various technical features and it should be understood that the embodiments presented herein are not limited by the accompanying drawings. As such, the present disclosure should be construed to extend to any alterations, equivalents, and substitutes in addition to those which are particularly set out in the accompanying drawings. Although the terms first, second, etc. may be used herein to describe various elements, these elements should not be limited by these terms. These terms are generally only used to distinguish one element from another.

Accordingly, the embodiments herein achieve a method for AI-based assistive health sensing in an IoT network comprising a plurality of electronic device connected with each other. The method includes obtaining, by a first electronic device from the plurality of electronic devices, at least one input indicating a current health condition of a user. Further, the method includes determining, by the first electronic device, at least one vital parameters of the user to be measured based on the current health conditions of the user using at least one AI model. Further, the method includes identifying, by the first electronic device, at least one second electronic device from the plurality of electronic devices to measure the at least one vital parameter for the user using the at least one AI model. Further, the method includes automatically initiating, by the first electronic device, a conversation with the user. The conversation includes an operating guidance to measure the at least one vital parameter of the user using the at least one second electronic device.

Unlike conventional methods and systems, the proposed method can be used to automatically enable sensors to measure the vital signs along with health parameters using the AI model and electronic devices associated with the user. Further, the proposed method can be used to prepare an assessment report for expert diagnosis with measured health parameters, the vital signs, and past EHR data using the AI model and the electronic devices. The proposed method can be used to provide a care plan prescribed by the doctor in an automatic manner in a smart home environment.

The method can be used to provide an AI based pre-consultation to a user (e.g., patient). Consider, the user is a very busy mother with a kid and is due for her second child. The mother has a busy routine with home, office, kid classes, and weekly doctor appointments. The mother is educated and uses online resources and comfortable using various electronic devices. The mother motive is to take care of themselves, her family's health and keep family healthy. When someone in the her family is unwell, the mother wants quick, convenient and reliable care, and need to avoid travel and only visit the doctor if it is absolutely necessary.

Consider the mother is not well, based on the proposed method, the mother feeds an input to a virtual assistance application executed in the electronic device as feeling dizzy and stressed. Based on the input, the virtual assistance application asks further question to the mother and triggers a medical knowledge analysis for monitoring an AI-based assistive health in the IoT network. Based on the monitoring, the electronic device automatically initiates a conversation with the mother to measure the vital parameter (e.g., BP level, or the like) at the predetermined time prior to the appointment with the caretaker.

Referring now to the drawings, and more particularly to FIGS. 1A through 7, where similar reference characters denote corresponding features consistently throughout the figures, there are shown preferred embodiments.

FIG. 1A illustrates overview of an IoT network (1000a) comprising a plurality of electronic devices (100a-100n) for AI-based assistive health sensing, according to the embodiments as disclosed herein. The IoT network (1000a) can be a smart home-based online healthcare system. In an embodiment, the IoT network (1000a) includes a plurality of electronic device (100a-100n) connected with each other. The electronic device (100a-100n) can be, for example, but not limited to a smart watch, a smart phone, a AI speaker, an IoT sensor, a laptop, a smart social robot, a Personal Digital Assistant (PDA), a tablet computer, a laptop computer, a music player, a video player, or the like.

In an embodiment, a first electronic device (100a) from the plurality of electronic device (100a-100n) is configured to obtain at least one input indicating a current health condition of a user. The current health condition can be, for example but not limited to feeling stressed, dizziness, vomiting sense, oxygen saturation, Blood Pressure (BP) values, electrocardiogram (ECG) readings, heart-rate or the like. The input can be, for example, but not limited to command, a text based command, a physical command, an IoT command, visual/perceptual user interface (PUI) gestures, or the like. The electronic device 100a handles any mode of input. In an embodiment, the input can be a multi-modal query, user command, and device initiated interaction with the user. The multi-modal query provides interaction with multiple modes of interacting with the electronic device (100a), such as gestures, speech, text, video, audio, etc.

Based on the current health conditions of the user, the first electronic device (100a) is configured to determine at least one vital parameters of the user to be measured using at least one AI model (not shown). The vital parameter can be, for example but not limited to weight of the user, a body temperature of the user, a brain activity of the user, a skin conductance of the user, a pulse rate of the user or the like.

Further, the first electronic device (100a) is configured to identify at least one another electronic device (100b-100n) from the plurality of electronic devices (100a-100n) to measure the at least one vital parameter for the user using the at least one AI model. In an embodiment, the at least one another electronic device is identified from the plurality of electronic devices (100a-100n) by determining a capability of each of the electronic devices (100b-100n) connected to the electronic device (100a).

Based on the identification, the first electronic device (100a) is configured to automatically initiate a conversation with the user. The conversation includes an operating guidance to measure the at least one vital parameter of the user using the at least one another electronic device (100b-100n). In an example, the first electronic device (100a) assists the doctor to measure the vital signs by giving access to the electronic devices (100b-100n) associated with the user, highlights summary details during the conversation, and also assist the user with a medical content in a display to understand the medical condition.

In an embodiment, the first electronic device (100a) is further configured to obtain the at least one measured vital parameter using the at least one another electronic device (100b-100n) for the user. Further, the first electronic device (100a) is configured to analyze the measured vital parameters. Further, the first electronic device (100a) is configured to recommend a caretaker related to the current health condition of the user for appointment based on the analysis. The caretaker can be, for example, but not limited to a doctor, a physician, or the like.

In an embodiment, the first electronic device (100a) is further configured to receive a health care instruction from the caretaker based on the at least one vital parameter. The health care instruction can be, for example, but not limited to a recommending a medical capsule for the user, suggesting for walking in a morning rime, or the like. Further, the first electronic device (100a) is configured to monitor the health care instruction based on the at least one vital parameter.

In an embodiment, the first electronic device (100a) is further configured to obtain at least one input indicating the current health condition of the user. Further, the first electronic device (100a) is configured to automatically book an appointment with the caretaker related to the current health condition of the user. Further, the first electronic device (100a) is configured to automatically initiate the conversation with the user to measure the at least one vital parameter at a predetermined time prior to the appointment with the caretaker. In an example, the AI model (e.g., AI voice agent, or the like) monitors the user's physiological parameters in communication with a server (e.g., cloud server, or the like). In an example, the AI model in communication with the server to process the user s physiological parameters. Further, the patient shares the health condition with the AI model. The AI model interacts with other electronic device 100b-100n or the server to automatically enable sensors to measure the vital signs along with other parameters. The AI model prepares the assessment report which will be shared to the doctor before consultation. The assessment report summarizes the real-time health parameters, current problems of the user and allergies of the user, etc. In an embodiment, previous physical EHRs are appended along with the assessment report without adding or making any changes to the assessment report.

In an embodiment, the first electronic device (100a) is further configured to determine a capability of each of the electronic devices connected to the first electronic device (100a). Further, the first electronic device (100a) is configured to identify the at least one another electronic device to measure the at least one vital parameter for the user based on the capability of each of the electronic devices using the at least one AI model. Further, the first electronic device (100a) is configured to initiate the conversation comprising the operating guidance to measure the at least one vital parameter of the user using the at least one another electronic device.

In an embodiment, the first electronic device (100a) is further configured to obtain the at least one measured vital parameter using the at least one another electronic device (100b-100n) for the user. Further, the first electronic device (100a) is configured to share the at least one measured vital parameter with the caretaker prior to the appointment.

In an embodiment, the appointment with the caretaker is automatically booked by recommending the caretaker related to the current health condition of the user and receiving the confirmation from the user for the appointment with the caretaker.

Consider an example, if the user converse with the AI speaker about the health symptoms. The AI speaker automatically selects a natural language processing (e.g., Open Health Natural Language Processing (OHNLP) or the like) for conversation with medical narrative to the user. Further, the AI speaker triggers two operations (e.g., automate vital sign measurement relevant to the symptoms using connected electronic devices and analyzes the EHR data and a lifestyle data and its relation with the symptoms and user inputs in background in parallel based on user inputs on the health condition and other attributes (ex. duration, severity etc.). The lifestyle data is captured a health application running in the electronic device (100a). The health application tracks various information (e.g., active time, food & sleep level of the user or the like).

Further, the AI speaker provides a reasoning of the users symptoms with knowledge bases augmented with the vital sign and EHR data. Further, the AI speaker predicts the probabilities of results (e.g., disease, health condition or the like). Further, the AI speaker applies a classification model on the results. Based on the classification model, the results are classified into a low level, average level and high level to book the appointment with the caretaker. Further, the AI speaker generates the assessment report about the user symptoms for the expert consultation (e.g. doctor consultation or the like). The assessment report is shared with the doctor before online consultation. The summary of the assessment report serves as a starting point for the diagnosis. The user connected other electronic devices control is provided to the doctor where the doctor gets access to the other electronic device (100b-100n) and can check additional parameters. Based on the doctor consultation, the AI speaker can add the consultation information (e.g., medical secure capsule suggestion, or the like) based on experts choice. This capsule displays medical information like images/video/graphic content to the user based on the conversation.

Further, the AI speaker creates a metadata based on the symptoms and diagnosis outcome which acts as cataloging of data sources, transformations, data lineage and relationships. Further, the AI speaker evaluates the predicted assessment report and doctor diagnosis summary and updates ontology learning. Based on the online consultation, the AI speaker handles the care plan management. In an example, when routines is accepted by the users, daily routine schedule is created, sets reminders, set medicine, food intake schedule etc., and alert mechanism in case of any abnormality.

FIG. 1B illustrates another overview of the IoT network (1000b) comprising the plurality of electronic device (100a-100n) and the server (200) for AI-based assistive health sensing, according to the embodiments as disclosed herein. In an embodiment, the IoT network (1000a) includes the plurality of electronic device (100a-100n) connected with each other and a server (200). The server (200) is communicated with the one or more electronic device (100a-100n). The operations and functions of the electronic device (100a-100n) is already explained in conjunction with the FIG. 1A.

In an embodiment, the first electronic device (100a) is configured to obtain the input indicating the current health condition of the user. Based on the current health conditions of the user, the first electronic device (100a) is configured to determine the vital parameters of the user to be measured using the server (200). The server (200) analyzes the current health conditions based on measured health parameters, the lifestyle data, the EHR data and the environmental condition.

In an embodiment, the real-time health parameters collected from the health sensor is converted into the global standard format (Fast Healthcare Interoperability Resources (FHIR) which is in JavaScript Object Notation (JSON) format currently) and passed to the server (200). The server (200) receives the health parameters and identifies the health issue based on the health parameters and provides the relationship with health issue and the health parameters.

In an example, the first electronic device (100a) monitors the users physiological parameters in communication with the server (e.g., cloud server, or the like). In an example, the first electronic device (100a) in communication with the server (200) to process the user s physiological parameters. Based on the processed user's physiological parameters, the first electronic device (100a) shares the health condition with the care taker or understands the user health condition.

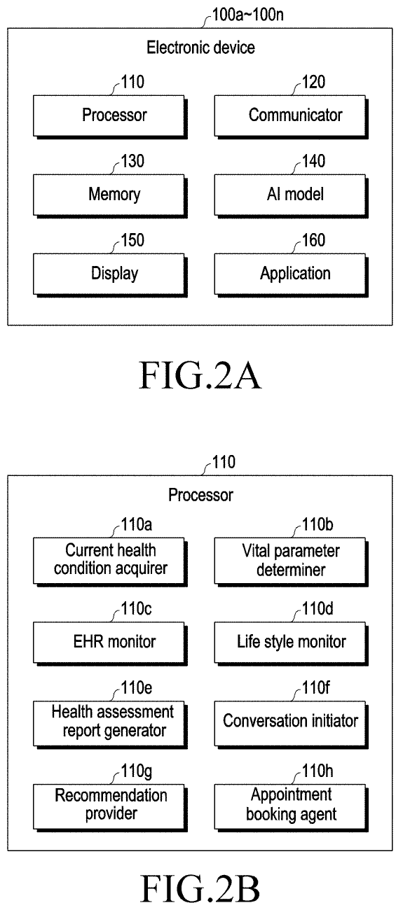

FIG. 2A illustrates various hardware blocks of the electronic device (100a-100n), according to the embodiments as disclosed herein. In an embodiment, the electronic device (100a-100n) includes a processor (110), a communicator (120), a memory (130), the AI model (140), a display (150) and an application (160). The processor (110) is coupled with the communicator (120), the memory (130), the AI model (140), the display (150) and the application (160). The application (160) can be, for example, but not limited to a virtual assistance application, a voice assistance application, a fitness related application, an IoT application, a health care application or the like. In an embodiment, the application (160) is connected to the AI model (140). In another embodiment, the AI model (140) resides in the application (160).

In an embodiment, the processor (110) is configured to obtain the input indicating the current health condition of the user using the application (160). Based on the current health conditions of the user, the processor (110) is configured to determine the vital parameters of the user to be measured using the AI model (140).

Further, the processor (110) is configured to identify another electronic device (100b-100n) from the plurality of electronic devices (100a-100n) to measure the vital parameter for the user using the AI model (140). Based on the identification, the processor (110) is configured to automatically initiate the conversation with the user. The conversation includes the operating guidance to measure the vital parameter of the user using the at least one another electronic device (100b-100n). The operating guidance is displayed on the display (150).

In an embodiment, the processor (110) is further configured to obtain the measured vital parameter using the at least one another electronic device (100b-100n) for the user. Further, the processor (110) is configured to analyze the measured vital parameters. Further, the processor (110) is configured to recommend the caretaker related to the current health condition of the user for appointment based on the analysis.

In an embodiment, the processor (110) is further configured to receive the health care instruction from the caretaker based on the vital parameter. Further, the processor (110) is configured to monitor the health care instruction based on the vital parameter.

In an embodiment, the processor (110) is further configured to obtain at least one input indicating the current health condition of the user. Further, the processor (110) is configured to automatically book the appointment with the caretaker related to the current health condition of the user. Further, the processor (110) is configured to automatically initiate the conversation with the user to measure the vital parameter at the predetermined time prior to the appointment with the caretaker.

In an example, the AI model (140) monitors the user s physiological parameters in communication with the server (200). In an example, the AI model (140) in communication with the server (200) to process the user's physiological parameters. Further, the patient shares the health condition with the AI model (140). The AI model interacts with other electronic device (100b-100n) or the server (200) to automatically enable sensors to measure the vital signs along with other parameters. The AI model (140) prepares the assessment report which will be shared to the doctor before consultation. The assessment report summarizes the real-time health parameters, active problems and allergies of the user.

In an embodiment, the processor (110) is further configured to determine the capability of each of the electronic devices (100b-100n) connected to the electronic device (100a). Further, the processor (110) is configured to identify the at least one another electronic device (100b-100n) to measure the at least one vital parameter for the user based on the capability of each of the electronic devices (100b-100n) using the AI model (140). Further, the processor (110) is configured to initiate the conversation comprising the operating guidance to measure the vital parameter of the user using the at least one another electronic device (100b-100n).

In an embodiment, the processor (110) is further configured to obtain the measured vital parameter using another electronic device (100b-100n) for the user. Further, the processor (110) is configured to share the measured vital parameter with the caretaker prior to the appointment.

In an embodiment, the appointment with the caretaker is automatically booked by recommending the caretaker related to the current health condition of the user and receiving the confirmation from the user for the appointment with the caretaker.

In an embodiment, the processor (110) is further configured to receive the health care instruction from the caretaker based on the vital parameter. Further, the processor (110) is configured to monitor the health care instruction based on the vital parameter.

In an embodiment, the processor (110) is further configured to obtain the input indicating the current health condition of the user. Further, the processor (110) is configured to automatically book the appointment with the caretaker related to the current health condition of the user. Further, the processor (110) is configured to automatically initiate the conversation with the user to measure the vital parameter at the predetermined time prior to the appointment with the caretaker.

The processor (110) is configured to execute instructions stored in the memory (130) and to perform various processes. The communicator (120) is configured for communicating internally between internal hardware components and with external devices via one or more networks.

The memory (130) stores instructions to be executed by the processor (110). The memory (130) may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory (130) may, in some examples, be considered a non-transitory storage medium. The term “non-transitory” may indicate that the storage medium is not embodied in a carrier wave or a propagated signal. However, the term “non-transitory” should not be interpreted that the memory (130) is non-movable. In some examples, the memory (130) can be configured to store larger amounts of information than the memory. In certain examples, a non-transitory storage medium may store data that can, over time, change (e.g., in Random Access Memory (RAM) or cache).

Although the FIG. 2A shows various hardware components of the electronic device (100a-100n) but it is to be understood that other embodiments are not limited thereon. In other embodiments, the electronic device (100a-100n) may include less or more number of components. Further, the labels or names of the components are used only for illustrative purpose and does not limit the scope of the invention. One or more components can be combined together to perform same or substantially similar function to handle the AI-based assistive health sensing in the IoT network (1000a and 1000b).

FIG. 2B illustrates various hardware blocks of the processor (110) included in the electronic device (100a-100n), according to the embodiments as disclosed herein. In an embodiment, the processor (110) includes a current health condition acquirer (110a), a vital parameter determiner (110b), an EHR monitor (110c), a life style monitor (110d), a health assessment report generator (110e), a conversation initiator (110f), a recommendation provider (110g), and an appointment booking agent (110h).

In an embodiment, the current health condition acquirer (110a) is configured to obtain the input indicating the current health condition of the user using the application (160). Based on the current health conditions of the user, the vital parameter determiner (110b) is configured to determine the vital parameters of the user to be measured using the AI model (140).

Further, the vital parameter determiner (110b) is configured to identify another electronic device (100b-100n) from the plurality of electronic devices (100a-100n) to measure the vital parameter for the user using the AI model (140). Based on the identification, the conversation initiator (110f) is configured to automatically initiate the conversation with the user.

In an embodiment, the vital parameter determiner (110b) is further configured to obtain the measured vital parameter using the at least one another electronic device (100b-100n) for the user. Further, the vital parameter determiner (110b) is configured to analyze the measured vital parameters. Further, the recommendation provider (110g) is configured to recommend the caretaker related to the current health condition of the user for appointment based on the analysis.

In an embodiment, the conversation initiator (110f) is further configured to receive the health care instruction from the caretaker based on the vital parameter. Further, the EHR monitor (110c) is configured to monitor the health care instruction based on the vital parameter.

In an embodiment, the vital parameter determiner (110b) is further configured to obtain at least one input indicating the current health condition of the user. Further, the appointment booking agent (110h) is configured to automatically book the appointment with the caretaker related to the current health condition of the user. Further, the conversation initiator (110f) is configured to automatically initiate the conversation with the user to measure the vital parameter at the predetermined time prior to the appointment with the caretaker.

In an embodiment, the vital parameter determiner (110b) is further configured to obtain the measured vital parameter using another electronic device (100b-100n) for the user. Further, the conversation initiator (110f) is configured to share the measured vital parameter with the caretaker prior to the appointment.

In an embodiment, the EHR monitor (110c) is further configured to receive the health care instruction from the caretaker based on the vital parameter. Further, the EHR monitor (110c) is configured to monitor the health care instruction based on the vital parameter.

The vital parameter determiner (110b) analyzes the current health conditions based on measured health parameters, the lifestyle data, the EHR data and the environmental condition using the EHR monitor (110c) and the life style monitor (110d).

Although the FIG. 2B shows various hardware components of the processor (110) but it is to be understood that other embodiments are not limited thereon. In other embodiments, the processor (110) may include less or more number of components. Further, the labels or names of the components are used only for illustrative purpose and does not limit the scope of the invention. One or more components can be combined together to perform same or substantially similar function to handle the AI-based assistive health sensing in the IoT network (1000a and 1000b).

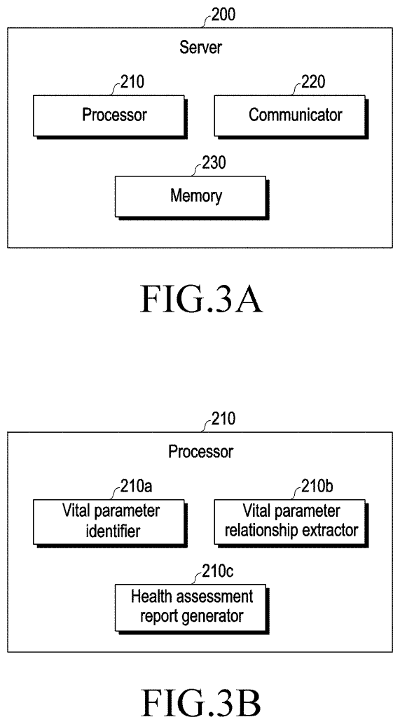

FIG. 3A illustrates various hardware blocks of the server (200), according to the embodiments as disclosed herein. In an embodiment, the server (200) includes a processor (210), a communicator (220), and a memory (230). The processor (210) is coupled with the communicator (220) and the memory (230).

In an embodiment, the processor (210) is configured to obtain the input indicating the current health condition of the user. Based on the current health conditions of the user, the processor (210) is configured to determine the vital parameters of the user to be measured. The processor (210) analyzes the current health conditions based on measured health parameters, the lifestyle data, the EHR data and the environmental condition.

The processor (210) is configured to execute instructions stored in the memory (230) and to perform various processes. The communicator (220) is configured for communicating internally between internal hardware components and with external devices via one or more networks.

The memory (230) stores instructions to be executed by the processor (210). The memory (230) may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory (230) may, in some examples, be considered a non-transitory storage medium. The term “non-transitory” may indicate that the storage medium is not embodied in a carrier wave or a propagated signal. However, the term “non-transitory” should not be interpreted that the memory (230) is non-movable. In some examples, the memory (230) can be configured to store larger amounts of information than the memory. In certain examples, a non-transitory storage medium may store data that can, over time, change (e.g., in Random Access Memory (RAM) or cache).

Although the FIG. 3A shows various hardware components of the server (200) but it is to be understood that other embodiments are not limited thereon. In other embodiments, the server (200) may include less or more number of components. Further, the labels or names of the components are used only for illustrative purpose and does not limit the scope of the invention. One or more components can be combined together to perform same or substantially similar function to handle the AI-based assistive health sensing in the IoT network (1000a and 1000b).

FIG. 3B illustrates various hardware blocks of the processor (210) included in the server (200), according to the embodiments as disclosed herein. In an embodiment, the processor (210) includes a vital parameter identifier (210a), a vital parameter relationship extractor (210b), and a health assessment report generator (210c).

In an embodiment, the vital parameter identifier (210a) is configured to obtain the input indicating the current health condition of the user. Based on the current health conditions of the user, the vital parameter identifier (210a) is configured to determine the vital parameters of the user to be measured using the vital parameter relationship extractor (210b). The health assessment report generator (210c) analyzes the current health conditions based on measured health parameters, the lifestyle data, the EHR data and the environmental condition and generates the report based on the measured health parameters, the lifestyle data, the EHR data and the environmental condition

Although the FIG. 2B shows various hardware components of the processor (210) but it is to be understood that other embodiments are not limited thereon. In other embodiments, the processor (210) may include less or more number of components. Further, the labels or names of the components are used only for illustrative purpose and does not limit the scope of the invention. One or more components can be combined together to perform same or substantially similar function to handle the AI-based assistive health sensing in the IoT network (1000a and 1000b).

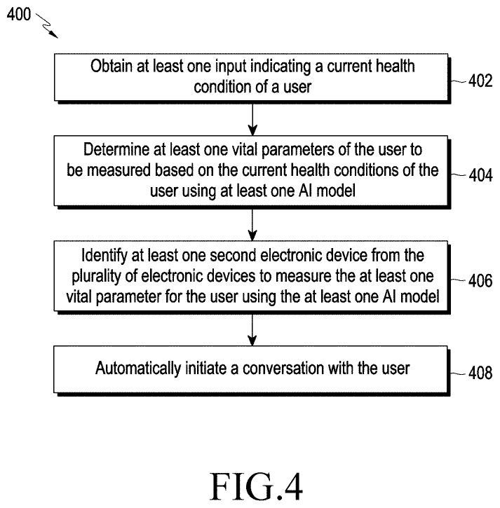

FIG. 4 illustrates a flow chart (400) for automatically initiating the conversation, with the user, comprising the operating guidance to measure the at least one vital parameter of the user, according to the embodiments as disclosed herein.

As shown in the FIG. 4, the operations (402-408) are performed by the processor (110). At 402, the method includes obtaining the at least one input indicating the current health condition of the user. At 404, the method includes determining the at least one vital parameters of the user to be measured based on the current health conditions of the user using at least one AI model (140). At 406, the method includes identifying the at least one second electronic device from the plurality of electronic devices (100a-100n) to measure the at least one vital parameter for the user using the at least one AI model (140). At 408, the method includes automatically initiating the conversation with the user. The conversation includes an operating guidance to measure the at least one vital parameter of the user using the at least one second electronic device (100b-100n).

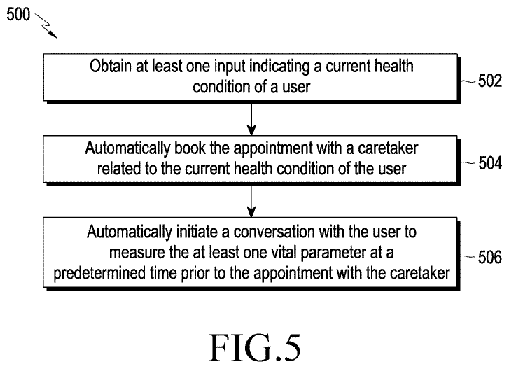

FIG. 5 illustrates a flow chart (500) for automatically initiating the conversation with the user to measure at least one vital parameter at the predetermined time prior to the appointment with the caretaker, according to the embodiments as disclosed herein.

As shown in the FIG. 5, the operations (502-506) are performed by the processor (210). At 502, the method includes obtaining the at least one input indicating the current health condition of the user. At 504, the method includes automatically booking the appointment with the caretaker related to the current health condition of the user. At 506, the method includes automatically initiating the conversation with the user to measure the at least one vital parameter at the predetermined time prior to the appointment with the caretaker.

The various actions, acts, blocks, steps, or the like in the flow charts (400 and 500) may be performed in the order presented, in a different order or simultaneously. Further, in some embodiments, some of the actions, acts, blocks, steps, or the like may be omitted, added, modified, skipped, or the like without departing from the scope of the invention.

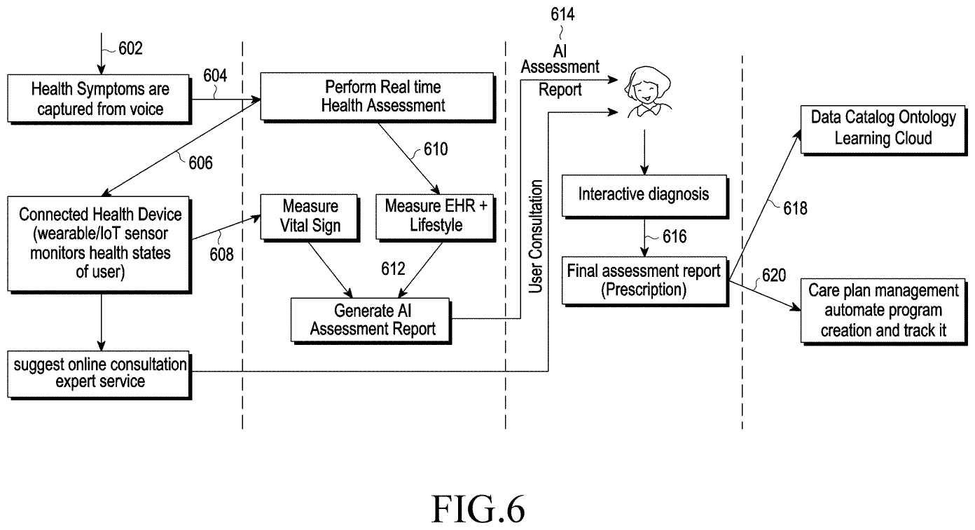

FIG. 6 illustrates are example sequence diagram including various operations for providing a care plan management based on the AI-based assistive health sensing in the IoT network (1000a-1000b), according to the embodiments as disclosed herein.

At 602, the AI speaker captures the health symptoms from the voice input from the user. At 604, the AI speaker performs the real time health assessment for the user. At 606, the AI speaker monitors health states of the user using the connected electronic devices. At 608 and 610, the AI speaker measures the vital sign of the user, the EHR of the user and the lifestyle information of the user. The AI speaker suggests the online consultation expert service for the user. At 612, the AI speaker generates the assessment report. At 614, the AI speaker shares the assessment report to the doctor. At 616, the doctor provides a final assessment report based on the interactive diagnosis. Based on the final assessment report, data catalog ontology learning is provided in the server (200) at 618. Based on the final assessment report, the AI speaker creates the care plan management automate procedure and tracks the user health based on the care plan management automate procedure at 620.

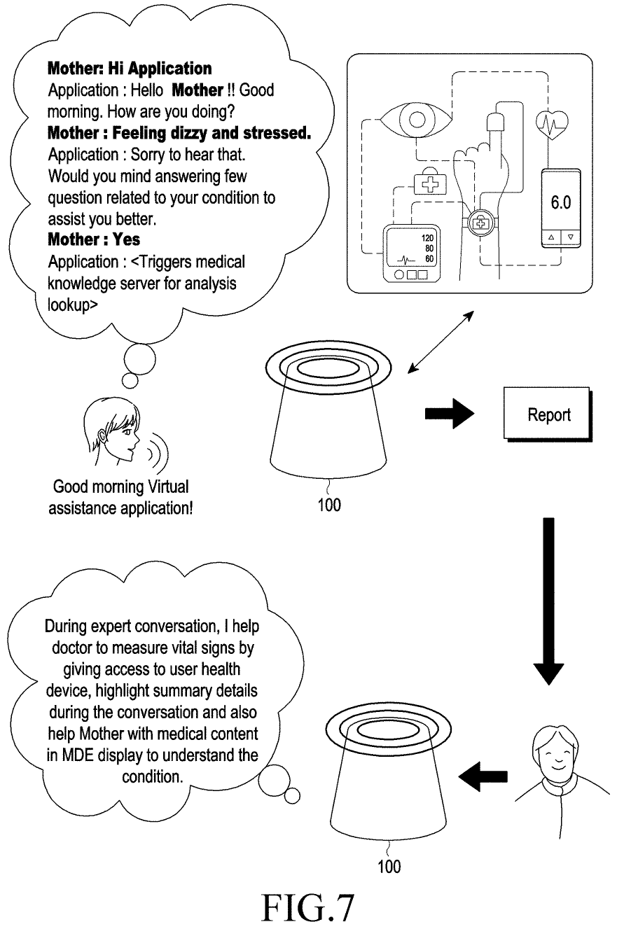

FIG. 7 illustrates an example in which the electronic device provides health care decision, according to the embodiments as disclosed herein.

Consider, the user is a very busy mother with a kid and is due for her second child. The mother has a busy routine with home, office, kid's classes and weekly doctor appointments. The mother is educated and uses online resources and comfortable using various electronic devices. The mother motive is to take care of themselves, her family's health and keep family healthy. When someone in the family is unwell, the mother wants quick, convenient and reliable care, and need to avoid travel and only visit the doctor if it is absolutely necessary.

Based on the proposed methods, the AI speaker provides voice based assistance based on symptoms and keywords received from the mother and checks for other electronic devices that are accessible from home or work. The AI speaker books the online appointments by analyzing the symptoms and the keywords in the electronic devices, creates routines and orders online medication. Further, the AI speaker connects to a family doctor/specialist from other city via chat or video call and also help in sharing the user's report.

In another example, Mother feeds the input to the virtual assistance application as feeling dizzy and stressed. Based on the input, the virtual assistance application asks further question to the mother and triggers medical knowledge analysis for monitoring the AI-based assistive health in the IoT network (1000a-1000b).

Based the conversation with mother, initial states of her condition are State of user (‘Dizziness’, ‘Stress’).

From the states, with the help of knowledge graph expected outcome are derived Possible outcomes=(‘Low Blood pressure’, ‘anemia’, ‘less sleep’).

Based on the existing knowledge base, states start with the following probabilities s1 and s2: Trigger Start={‘Dizziness’: s1, ‘Stress’: s2}.

The AI speaker will trigger graph based data/medical knowledge database to determining if trigger t1, t2 and t3 will need doctor's assistance.

trigger = {

```
  {‘Low Blood pressure ′: t1,},
  {‘anemia’: t2}
  {‘Lack of Sleep′: t3}
}
```

With help of relational reasoning network (Text/Image) weightage is assigned to causes (s1t1, s2t2, s1t3)=w1 & (s2t1, s2t2, s3t3)=w2

Weightage W1 = {‘Dizziness^{′}: s1

{‘Low Blood pressure ’: t1}
{‘anemia': t2}
{'Lack of sleep’: t3}

}
Weightage W2 = {‘Stress': s2
{‘Low Blood pressure ’: t1}
{‘anemia’: t2}
{‘Lack of sleep’: t3}
}

If dizziness with low blood pressure is giving higher joint weightage, the reason for fatigue resulting in doctor appointment is predicted. The summary report is prepared with the relevant evidence of low blood pressure being the major cause for mother condition and highlights the mother is pregnant currently. The key finding and vital sign information are captured in the assessment report for expert review and is passed to the doctor.

During the expert conversation, the mother assists the doctor to measure vital signs by giving the doctor access to user health device, highlighting summary details during the conversation and also helps the mother with medical content in MDE display to understand the condition. The push care plan set by the doctor to the individual tracking devices.

The expert can set medical/programs routines to the mother. The set routines are pushed to the mother after consultation. Upon accepting the routines by the mother, a daily routine schedule is created in the AI speaker, sets reminders, set medicine, food intake schedule etc. in the AI speaker and alert mechanism is initiated in the AI speaker in case of any abnormality.

This capsule displays medical information like images/video/graphic content to the mother based on conversation. For example: When the doctor explains about a baby in a womb, content helps the mother to visualize in a better way. Choose right communication for MDE. The doctor can set medical/programs routines to the mother. The set routines will be pushed to the mother after consultation.

The embodiments disclosed herein can be implemented using at least one software program running on at least one hardware device and performing network management functions to control the elements.

Although the present disclosure has been described with various embodiments, various changes and modifications may be suggested to one skilled in the art. It is intended that the present disclosure encompass such changes and modifications as fall within the scope of the appended claims.

What is claimed is:

1. A method for artificial intelligence (AI)-based assistive health sensing in an internet of things (IoT) network comprising a plurality of electronic devices connected with each other, comprising:
obtaining, by a first electronic device from the plurality of electronic devices, at least one input indicating a current health condition of a user;
determining, by the first electronic device, at least one vital parameter of the user to be measured based on the current health condition of the user using at least one AI model;
identifying, by the first electronic device, at least one second electronic device from the plurality of electronic devices to measure the at least one vital parameter for the user using the at least one AI model; and
automatically initiating, by the first electronic device, a conversation with the user, wherein the conversation comprises an operating guidance to measure the at least one vital parameter of the user using the at least one second electronic device.

2. The method of claim 1, wherein identifying, by the first electronic device, the at least one second electronic device from the plurality of electronic devices to measure the at least one vital parameter for the user using the at least one AI model comprises:
determining, by the first electronic device, a capability of each of the plurality of electronic devices connected to the first electronic device; and
identifying, by the first electronic device, the at least one second electronic device to measure the at least one vital parameter for the user based on the capability of each of the plurality of electronic devices.

3. The method of claim 1, further comprises:
obtaining, by the first electronic device, the at least one vital parameter of the user from the at least one second electronic device;
analyzing, by the first electronic device, the at least one vital parameter obtained from the at least one second electronic device; and
recommending, by the first electronic device, a caretaker related to the current health condition of the user based on the analysis.

4. The method of claim 3, further comprises:
after recommending the caretaker, transmitting the at least one vital parameter to the caretaker; and
connecting the user with the caretaker for a consultation.

5. The method of claim 1, further comprises:
receiving, by the first electronic device, a health care instruction from a caretaker based on the at least one vital parameter; and
monitoring, by the first electronic device, the health care instruction based on the at least one vital parameter.

6. A method for artificial intelligence (AI)-based assistive health sensing in an internet of things (IoT) network comprising a plurality of electronic devices connected with each other, comprising:
obtaining, by a first electronic device from the plurality of electronic devices, at least one input indicating a current health condition of a user;
automatically booking, by the first electronic device, an appointment with a caretaker related to the current health condition of the user; and
automatically initiating, by the first electronic device, a conversation with the user to measure at least one vital parameter at a predetermined time prior to the appointment with the caretaker.

7. The method of claim 6, further comprises:
determining, by the first electronic device, a capability of each of the plurality of electronic devices connected to the first electronic device;
identifying, by the first electronic device, at least one second electronic device to measure the at least one vital parameter for the user based on the capability of each of the plurality of electronic devices using at least one AI model; and
initiating, by the first electronic device, the conversation comprising an operating guidance to measure the at least one vital parameter of the user using the at least one second electronic device.

8. The method of claim 7, further comprises:
obtaining, by the first electronic device, the at least one vital parameter using the at least one second electronic device for the user; and
sharing, by the first electronic device, the at least one vital parameter with the caretaker prior to the appointment.

9. The method of claim 6, wherein automatically booking, by the first electronic device, the appointment with the caretaker related to the current health condition of the user comprises:
recommending by the first electronic device, the caretaker related to the current health condition of the user;
receiving, by the first electronic device, a confirmation from the user for the appointment with the caretaker; and
booking, by the first electronic device, the appointment with the caretaker.

10. The method of claim 9, further comprises:
receiving, by the first electronic device, a health care instruction from the caretaker based on the at least one vital parameter; and
monitoring, by the first electronic device, the health care instruction based on the at least one vital parameter.

11. An electronic device for artificial intelligence (AI)-based assistive health sensing in an internet of things (IoT) network comprising a plurality of electronic devices connected with each other, comprising:
a memory;
at least one AI model; and
a processor, coupled with the memory and the at least one AI model, configured to:
obtain at least one input indicating a current health condition of a user;
determine at least one vital parameter of the user to be measured based on the current health condition of the user using the at least one AI model;
identify at least one second electronic device from the plurality of electronic devices to measure the at least one vital parameter for the user using the at least one AI model; and
automatically initiate a conversation with the user, wherein the conversation comprises an operating guidance to measure the at least one vital parameter of the user using the at least one second electronic device.

12. The electronic device of claim 11, wherein to identify the at least one second electronic device from the plurality of electronic devices to measure the at least one vital parameter for the user using the at least one AI model, the processor is configured to:
determine a capability of each of the plurality of electronic devices connected to the electronic device; and
identify the at least one second electronic device to measure the at least one vital parameter for the user based on the capability of each of the plurality of electronic devices.

13. The electronic device of claim 11, wherein the processor is further configured to:
obtain the at least one vital parameter of the user from the at least one second electronic device;
analyze the at least one vital parameter obtained from the at least one second electronic device; and
recommend a caretaker related to the current health condition of the user based on the analysis.

14. The electronic device of claim 13, wherein the processor is further configured to:
after recommending the caretaker to the user, transmit the at least one vital parameter to the caretaker; and
connect the user with the caretaker for a consultation.

15. The electronic device of claim 11, wherein the processor is further configured to:
receive a health care instruction from a caretaker based on the at least one vital parameter; and
monitor the health care instruction based on the at least one vital parameter.

16. An electronic device for artificial intelligence (AI)-based assistive health sensing in an internet of things (IoT) network comprising a plurality of electronic devices connected with each other, comprising:
a memory; and
a processor, coupled with the memory, configured to:
obtain at least one input indicating a current health condition of a user;
automatically book an appointment with a caretaker related to the current health condition of the user; and
automatically initiate a conversation with the user to measure at least one vital parameter at a predetermined time prior to the appointment with the caretaker.

17. The electronic device of claim 16, wherein the processor is further configured to:
determine a capability of each of the plurality of electronic devices connected to the electronic device;
identify at least one second electronic device to measure the at least one vital parameter for the user based on the capability of each of the plurality of electronic devices using at least one AI model; and
initiate the conversation comprising an operating guidance to measure the at least one vital parameter of the user using the at least one second electronic device.

18. The electronic device of claim 17, wherein the processor is further configured to:
obtain the at least one vital parameter using the at least one second electronic device for the user; and
share the at least one vital parameter with the caretaker prior to the appointment.

19. The electronic device of claim 16, wherein to automatically book the appointment with the caretaker related to the current health condition of the user the processor is configured to:
recommend the caretaker related to the current health condition of the user;
receive a confirmation from the user for the appointment with the caretaker; and
book the appointment with the caretaker.

20. The electronic device of claim 19, wherein the processor is configured to:
receive a health care instruction from the caretaker based on the at least one vital parameter; and
monitor the health care instruction based on the at least one vital parameter.
